(19) United States
(12) Patent Application Publication
(10) Pub. No.: US 2022/0192936 A1
(43) Pub. Date: Jun. 23, 2022

(54) COMPOSITION FOR SOLID COSMETIC WITH HIGH-TEMPERATURE STABILITY AND IMPROVED FEELING OF USE AND PREPARATION METHOD THEREOF

(71) Applicant: COSMECCA KOREA CO., LTD., Chungcheongbuk-do (KR)

(72) Inventors: Hong Seok SON, Seongnam-si (KR); Won Seuk CHOI, Yongin-si (KR); Da Seul KIM, Suwon-si (KR); Hye Jin KIM, Seongnam-si (KR); Tae Hoon KIM, Suwon-si (KR); Young Sung YOO, Siheung-si (KR); Hyun Dae CHO, Cheongju-si (KR)

(73) Assignee: COSMECCA KOREA CO., LTD., Chungcheongbuk-do (KR)

(21) Appl. No.: 17/561,090

(22) Filed: Dec. 23, 2021

(30) Foreign Application Priority Data
Dec. 23, 2020 (KR) 10-2020-0182168

(51) Int. Cl. A61K 8/06; A61K 8/02; A61K 8/42; A61K 8/19; A61K 8/29; A61K 8/891; A61K 8/37; A61K 8/92; A61K 8/86; A61Q 1/02

(52) U.S. Cl. CPC A61K 8/064; A61K 8/0216; A61K 8/42; A61K 8/19; A61K 8/29; A61K 2800/43; A61K 8/375; A61K 8/92; A61K 8/86; A61Q 1/02; A61K 2800/24; A61K 8/891

(57) ABSTRACT

A composition for a solid cosmetic with improved high-temperature stability and good feeling of use, the composition contains an oily gelling agent. The oily gelling agent includes at least one selected from the group consisting of dibutylethylhexanoylglutamide, dibutyllauroylglutamide, dextrin palmitate, and mixtures thereof. When the oily gelling agent is dibutylethylhexanoylglutamide, the dibutylethylhexanoylglutamide is contained in an amount of 0.5 wt % to 1.7 wt % relative to the total weight of the composition.

### CROSS REFERENCE TO RELATED APPLICATION

The present application claims priority to Korean Patent Application No. 10-2020-0182168 (filed on Dec. 23, 2020), which is hereby incorporated by reference in its entirety.

### BACKGROUND

The present disclosure relates to a composition for a solid cosmetic with improved high-temperature stability and a good feeling of use and to a method for preparing the same. More particularly, the present disclosure relates to a composition for a solid cosmetic the structure of which can be maintained even at a high temperature, thereby having high-temperature stability, and which allows soft and easy application to the skin without giving a stuffy feeling. The present disclosure also relates to a method of preparing the same composition.

Women's desire to pursue beauty is not a thing of the past, and rather, interest in fitness, beauty, or cosmetics to make themselves look more beautiful is increasing.

As not only women but also men are interested in cosmetics for skin care, the cosmetics market continues to expand, and various types of cosmetics are being launched on the market.

In general, cosmetics can be largely divided into basic cosmetics, makeup bases, and color cosmetics all of which can b prepared as solids, liquids, or gels. There are also various types of cosmetic containers for storing them.

Recently, functional cosmetics having various functions such as moisturizing, whitening, wrinkle improvement, UV protection, acne relief, atopic relief, anti-inflammatory, and keratin dissolution are also widely consumed.

Among various cosmetics, solid cosmetics require molding conforming to the shapes of containers. In particular, containers for solid cosmetics and UV protection products are usually divided into balm types and stick types. In order for cosmetic containers to contain solid cosmetics, the cosmetics need to have an appropriate hardness.

For general solid balm-type cosmetics, consumers apply them to the skin by paying off them with a hand or a puff. Therefore, solid balm-type cosmetic products need to have a hardness to the extent that consumers can pay off them for use. When the hardness of the contents of a solid cosmetic product is insufficient, the contents will collapse when an external force is applied thereto. On the contrary, when the hardness of the contents of a solid cosmetic product is excessively high, the payoff will not work and the cosmetic cannot be applied to the skin.

A grinding compact powder container is configured such that a rotating cutting blade scrapes and discharges the contents (i.e., cosmetic) stored in the container. Unlike general compact powder containers, since external physical force is applied to the contents, the contents need to withstand physical stress as well as thermal stress at high temperatures.

Most of the general solid cosmetics are oil-dispersion formulations in which oily components are dispersed or water-in-oil formulations. An oily structuring agent is used to adjust the hardness of the formulations.

The oily-phase structuring agent uses polar wax, hydrocarbon non-polar wax, silicone non-polar wax, or silicone polymer. The polar wax is a wax containing one or more heterogeneous elements such as oxygen, nitrogen, silicone, or phosphorus. The hydrocarbon non-polar wax is a wax containing only carbon atoms and hydrogen atoms and containing no heteroatoms such as oxygen, nitrogen, silicon or phosphorus. The silicone non-polar wax contains silicon heteroatoms.

Solid cosmetics should not collapse even at a high temperature of about 50° C. and should not sweat to be a stable product. To increase the high-temperature stability of solid cosmetics, the content of the structuring agent may be increased or the content of the structuring agent with a high melting point may be increased. However, these methods have a problem in that the cosmetic is waxy, the application thereof is stiff, and the feeling of use is stuffy.

These days, consumers prefer a thin, soft, moist, and soft finish. In particular, in the era of wearing a mask due to COVID-19, natural makeup rather than a thick makeup is becoming a trend.

Therefore, in response to this demand, a composition for a solid cosmetic with high-temperature stability and good feeling of use is required. In addition, the solid cosmetic needs to maintain its structure even at a high temperature and needs to be lightly applied not to a give stuffy feeling.

### SUMMARY

Embodiments of the present disclosure are to maintain the structure of the contents of a cosmetic product even at a high temperature of about 50° C. by increasing the melting point of cosmetics and to ensure high-temperature stability of the cosmetics.

In addition, an objective of the present disclosure is to provide a cosmetic that can withstand not only thermal stress at high temperatures but also physical stress caused by external forces.

In addition, another objective of the present disclosure is to provide a cosmetic that can be normally shaved and discharged by a rotating cutting blade at a high temperature even when the cosmetic is applied to a grinding container.

In addition, a further objective of the present disclosure is to provide a cosmetic that can be applied lightly without heavy feeling while having an increased hardness compared to existing cosmetics.

In addition, a yet further objective of the present disclosure is to give a thin and soft sensation when the cosmetic is applied to the skin, thereby providing a natural skin expression which is pursued in a recent makeup trend.

In addition, a yet further objective of the present disclosure is to increase the degree of freedom in designing a cosmetic composition having an appropriate hardness with the use of a small amount of a structuring agent instead of existing structuring agents composed only of wax.

According to one aspect of the present disclosure, there is provided a solid cosmetic composition containing an oily gelling agent, the solid cosmetic composition having improved high-temperature stability and a good feeling of use.

The oily gelling agent may be made of at least one selected from the group consisting of dibutylethylhexanoylglutamide, dibutyllauroylglutamide, dextrin palmitate, and mixtures thereof.

When the oily gelling agent is dibutylethylhexanoylglutamide, the dibutylethylhexanoylglutamide may be contained in an amount of 0.5 wt % to 1.7 wt % relative to the total weight of the cosmetic composition.

When the oily gelling agent is dibutyllauroylglutamide, the dibutyllauroylglutamide may be contained in an amount of 0.5 wt % to 1.7 wt % relative to the total weight of the cosmetic composition.

When the oily gelling agent is a mixture of dibutylethylhexanoylglutamide and dibutyllauroylglutamide, the dibutylethylhexanoylglutamide may be contained in an amount of 0.25 wt % to 0.85 wt % relative to the total weight of the cosmetic composition, and the dibutyllauroylglutamide may be contained in an amount of 0.25 wt % to 0.85 wt % relative to the total weight of the cosmetic composition.

The oily gelling agent may be prepared by using octyldodecanol that is an aliphatic alcohol as a solvent.

The content of a solute of the oily gelling agent may be 5 to 9 times the content of a solvent, and the solute may be configured to be dissolved at a temperature of 110° C. to 120° C.

The solid cosmetic composition with improved high-temperature stability and good feeling of use, according to the present disclosure, may further contain an oil-soluble component, in which the oil-soluble component may be composed of at least one selected from the group consisting of fat, wax, hydrocarbon, natural oil, ester, hydrocarbon, silicone, organic UV absorbers, and mixtures thereof.

The oil-soluble component may be included in an amount of 20% to 60% relative to the total weight of the cosmetic composition.

When the solid cosmetic composition is of a water-in-oil type, an emulsifier included in the composition may be at least one selected from the group consisting of cetyl PEG/PPG-10/1 dimethiocone, PEG-10 dimethicone/vinyl dimethicone crosspolymer, polyglyceryl-3 polydimethylsiloxyethyl dimethicone, polyglyceryl-3 polyricinoleate, polyglyceryl-3 diisostearate, and mixtures thereof.

The cetyl PEG/PPG-10/1 dimethiocone may be and the PEG-10 dimethicone/vinyldimethicone crosspolymer may be contained in an amount of 0.2 wt % to 0.8 wt % and an amount of 0.2 wt % to 0.4 wt %, respectively.

The polyglyceryl-3 polydimethylsiloxyethyl dimethicone may be contained in an amount of 0.2 wt % to 0.8 wt %.

The polyglyceryl-3 polyricinoleate and the polyglyceryl-3 diisostearate may be contained in an amount of 0.4 wt % to 0.8 wt % and an amount of 0.1 wt % to 0.2 wt %, respectively.

According to another aspect of the present disclosure, there is provided a method of preparing a composition for a water-in-oil-type solid cosmetic with improved high-temperature stability and good feeling of use, the method including: primarily mixing an oily gelling agent, aliphatic alcohol, ester oil, and a pigment base that are oily components at a stirring rate of 900 rpm to 1100 rpm for a first predetermined time, using a mixer; secondarily mixing the mixture resulting from the primary mixing at a temperature of 75° C. to 80° C. at a stirring rate of 1400 rpm to 1600 rpm for a second predetermined time, using a homomixer, and adding a water-soluble component to the mixture to prepare an emulsion; and adding a powder component including a pigment and an additive to the emulsion and stirring the resulting mixture at a rate of 1400 rpm to 1600 rpm for a third predetermined time.

An emulsifier used to prepare the emulsion may include at least one selected from the group consisting of cetyl PEG/PPG-10/1 dimethiocone, PEG-10 dimethicone/vinyl dimethicone crosspolymer, polyglyceryl-3 polydimethylsiloxyethyl dimethicone, polyglyceryl-3 polyricinoleate, polyglyceryl-3 diisostearate, and mixtures thereof.

According to another aspect of the present disclosure, there is provided a method of preparing a composition for a solid cosmetic with high-temperature stability and improved feeling of use, the method including: mixing an oily gelling agent, aliphatic alcohol, ester oil, and a pigment base that are oily components for a first predetermined time at a stirring rate of 900 rpm to 1100 rpm using a mixer at a high temperature; further mixing the resulting mixture of the oily components at a temperature of 75° C. to 80° C. at a stirring rate of 1400 rpm to 1600 rpm for a second predetermined time using a homomixer; and adding a pigment, a powder component, and additive to the resulting mixture and mixing at a stirring rate of 1400 rpm to 1600 rpm for a third predetermined time.

Embodiments of the present disclosure relate a cosmetic that can maintain the structure thereof even at a high temperature of about 50° C. due to the increased melting point, thereby ensuring high-temperature stability.

In addition, an objective of the present disclosure is to provide a cosmetic that can withstand not only thermal stress at high temperatures but also physical stress caused by external forces.

In addition, another objective of the present disclosure is to provide a cosmetic that can be normally shaved and discharged by a rotating cutting blade at a high temperature even when the cosmetic is contained in a grinding container.

In addition, a further objective of the present disclosure is to provide a cosmetic that can be applied lightly without stuffy feeling while having an increased hardness compared to existing cosmetics.

In addition, a yet further objective of the present disclosure is to provide a cosmetic that can give a thin and soft sensation when the cosmetic is applied to the skin, thereby providing a natural skin expression which is pursued in a recent makeup trend.

In addition, a yet further objective of the present disclosure is to increase the degree of freedom in designing a cosmetic composition having an appropriate hardness with the use of a small amount of a gelling agent instead of existing structuring agents composed only of wax.

### DETAILED DESCRIPTION

Herein below, preferred embodiments of the present disclosure will be described in detail with reference to the accompanying drawings. However, the present invention is not limited to the embodiments described herein and may be embodied in other forms. The embodiments described herein are provided so that the present disclosure can be made thorough and complete and that the spirit of the present disclosure can be fully conveyed to those skilled in the art. Throughout the drawings and description of the embodiments, like components are designated by like reference numerals.

A solid cosmetic composition having improved high-temperature stability and good feeling of use, according to an embodiment of the present disclosure, contains an oily gelling agent.

The oily gelling agent may include at least one selected from the group consisting of dibutylethylhexanoylglutamide, dibutyllauroylglutamide, dextrin palmitate, and mixtures thereof.

When the oily gelling agent is dibutylethylhexanoylglutamide, the dibutylethylhexanoylglutamide may be contained in an amount of 0.5 wt % to 1.7 wt % relative to the total weight of the cosmetic composition.

When the oily gelling agent is dibutyllauroylglutamide, the dibutyllauroylglutamide may be contained in an amount of 0.5 wt % to 1.7 wt % relative to the total weight of the cosmetic composition.

When the oily gelling agent is a mixture of dibutylethylhexanoylglutamide and dibutyllauroylglutamide, the dibutylethylhexanoylglutamide may be contained in an amount of 0.25 wt % to 0.85 wt % relative to the total weight of the cosmetic composition, and the dibutyllauroylglutamide may be contained in an amount of 0.25 wt % to 0.85 wt % relative to the total weight of the cosmetic composition.

The oily gelling agent may be prepared by using octyldodecanol that is an aliphatic alcohol as a solvent.

The content of a solute of the oily gelling agent may be 5 to 9 times the content of a solvent, and the solute may be configured to be dissolved at a temperature of 110° C. to 120° C.

The solid cosmetic composition with improved high-temperature stability and good feeling of use, according to the present disclosure, may further contain an oil-soluble component, in which the oil-soluble component may be composed of at least one selected from the group consisting of fat, wax, hydrocarbon, natural oil, ester, hydrocarbon, silicone, organic UV absorbers, and mixtures thereof.

The oil-soluble component may be included in an amount of 20% to 60% relative to the total weight of the cosmetic composition.

The solid cosmetic composition with improved high-temperature stability and good feeling of use can be prepared by a method described below.

First, when the solid cosmetic is of a water-in-oil type, the following steps are performed:
mixing an oily gelling agent, aliphatic alcohol, ester oil, pigment base, etc., which are oily components for a first predetermined time at a stirring rate of 900 rpm to 1100 rpm using a mixer at a high temperature;
adding a water-soluble component to the mixture of the oily components to prepare an emulsion while mixing the mixture of the oily components at a high temperature of 75° C. to 80° C. using a homomixer at a stirring rate of 1400 rpm to 1600 rpm for a second predetermined time; and
adding a powder component including a pigment and an additive to the water-in-oil emulsion and mixing at a stirring rate of 1400 rpm to 1600 rpm for a third predetermined time.

In particular, in the case of a water drop type among the water-in-oil types, a single emulsifier component or two or less emulsifiers are used in a small amount.

Examples of the emulsifier include a PEG-based emulsifier and a polyglyceryl-based emulsifier. Among them, PEG-based emulsifiers include cetyl PEG/PPG-10/1 dimethicone and PEG-10 dimethicone/vinyl dimethicone crosspolymer. The polyglyceryl-based emulsifiers include polyglyceryl-3 polydimethylsiloxyethyl dimethicone, polyglyceryl-3 polyricinoleate, and polyglyceryl-3 diisostearate.

When the solid cosmetic is of an oil dispersion type, the following steps are performed:
mixing an oily gelling agent, aliphatic alcohol, ester oil, pigment base, etc., which are oily components, are mixed for a first predetermined time at a stirring rate of 900 rpm to 1100 rpm using a mixer at a high temperature;
further mixing the resulting mixture obtained through the previous mixing at a high temperature of 75° C. to 80° C. using a homomixer at a stirring rate of 1400 rpm to 1600 rpm for a second predetermined time; and
adding a pigment, a powder component, and an additive to the mixture obtained through the previous mixing and further mixing at a stirring rate of 1400 rpm to 1600 rpm for a third predetermined time.

Tables 1 and 2 below relate to cycle test conditions and standards and methods for measuring hardness.

TABLE 1

| Conditions | Test conditions | Note |
|---|---|---|
| Cycle | −4° C./25° C./44° C./25° C. | 1 cycle = |
| | 23 hours for each condition, | 96 hours, 4 days |
| | temperature change 1 hour) | |

TABLE 2

| | Measurement | |
|---|---|---|
| Classification | standard | Measurement method |
| Hardness | 2Φ, 5 mm, 2 | Taking out samples stabilized |
| | cm/min | at 25° C. for 6 hours and |
| | | Perform measurement with |
| | | Rheometer RTC-3002D |

### EXAMPLE 1, EXAMPLE 2, EXAMPLE 3, AND COMPARATIVE EXAMPLE 1

Gelling agents of Examples 1, 2, 3 and Comparative Example 1 were prepared according to the compositions shown in Table 3 below. Each of the mixtures was prepared by dissolving a solute at a high temperature (90° C. to 140° C.) by setting the solute-to-solvent ratio to 1:9.

TABLE 3

| | | | | | Comparative |
|---|---|---|---|---|---|
| Classification | Name of component | Example 1 | Example 2 | Example 3 | Example 1 |
| Gelling agent | Dibutylethylhexanoyl | 5.00 | 10.0 | | |
| | glutamide | | | | |
| | Dibutyllauroyl | 5.00 | | 10.0 | |
| | glutamide | | | | |
| | Dextrin palmitate | | | | 10.0 |
| Solvent | Octyldodecanol | 90.0 | 90.0 | 90.0 | 90.0 |

The dissolution temperature, melting point, and hardness of each of the mixtures were checked immediately after the preparation, according to the type of the gelling agent dissolved in the aliphatic alcohol in each of Examples 1, 2, 3 and Comparative Example 1. The results are shown in Table 4 below.

TABLE 4

| | Dissolution | Melting point | |
|---|---|---|---|
| Classification | temperature | of mixture | Hardness |
| Example 1 | 130° C. or | 100° C. or | 90 or more |
| | higher | higher | |
| Example 2 | 130° C. | 100° C. or | 90 |
| | | higher | |
| Example 3 | 120° C. | 75° C. to 80° C. | 60 |
| Comparative | 90° C. | 55° C. to 60° C. | 40 |
| Example 1 | | | |

From the results in Table 4, the dissolution temperature, melting point, and hardness of each mixture can be confirmed for each type of gelling agent. When the melting point of the mixture is 100° C. or higher, purified water or volatile components cannot be added during the preparation. In this case, the process is inevitably limited. Therefore, the temperature for the high-temperature process enabling proper preparation is 85° C. or lower. When taking into account such a temperature condition, the gelling agents of Example 3 and Comparative Example 1 are appropriate. When the hardness values of Example 3 and Comparative Example 1 are compared, it can be confirmed that the mixture of Example 3 exhibits a higher hardness.

### EXAMPLE 3 AND COMPARISON EXAMPLES 2 TO 3

Mixtures of Example 3 and Comparative Examples 2 and 3 were prepared according to the compositions shown in Table 5 below. Each of the mixtures was prepared by dissolving a solute at a high temperature (100° C. or higher) by setting a solute-to-solvent ratio to 1:9

TABLE 5

| | | Example | Comparative | Comparative |
|---|---|---|---|---|
| Classification | Name of component | 3 | Example 2 | Example 3 |
| Gelling agent | Dibutyllauroyl | 10.0 | 10.0 | 10.0 |
| | glutamide | | | |
| Solvent | Octyldodecanol | 90.0 | | |
| | Ethylhexyl palmitate | | 90.0 | |
| | Cyclopentasiloxane | | | 90.0 |

Aside from aliphatic alcohol used as a solvent in Example 3, ester oil was used in Comparative Example 2 and silicone oil was used in Comparative Example 3. The dissolution temperature, melting point and hardness of each of the mixtures were checked immediately after the preparation thereof. The results are shown in Table 6 below.

TABLE 6

| | Dissolution | Melting point | |
|---|---|---|---|
| Classification | temperature | of mixture | Hardness |
| Example 3 | 120° C. | 75° C. to 80° C. | 60 |
| Comparative | 130° C. | 100° C. or | 115 |
| Example 2 | | higher | |
| Comparative | 150° C. or | — | — |
| Example 3 | higher | | |

The results of Table 6 show that the mixture prepared according to Comparative Example 2 exhibits a sufficiently high hardness but has an excessively high melting point and the mixture prepared according to Comparative Example 3 exhibits an excessively high dissolution temperature which may cause the solvent to be volatilized. Therefore, the solvent used in Comparative Example 3 is not suitable. Therefore it is confirmed that the aliphatic alcohol used in Example 3 is suitable as the solvent.

### EXAMPLES 4 TO 9

Mixtures of Examples 4 and 9 were prepared according to the compositions shown in Table 7 below. When preparing each of the mixtures, necessary components were dissolved at 100° C. or higher.

TABLE 7

| | Ex- | Ex- | Ex- | Ex- | Ex- | Ex- |
|---|---|---|---|---|---|---|
| Name of | ample | ample | ample | ample | ample | ample |
| component | 4 | 5 | 6 | 7 | 8 | 9 |
| Dibutyllauroyl | 10.0 | 10.0 | 10.0 | 10.0 | 10.0 | 10.0 |
| glutamide | | | | | | |
| Octyldodecanol | 90.0 | 80.0 | 70.0 | 60.0 | 50.0 | 40.0 |

Examples 4 to 9 were to find the most suitable gelling agent and the most suitable solute-to-solvent ratio. The melting points of the mixtures according to the content of the solvent are shown in Table 8 below.

TABLE 8

| | Ex- | Ex- | Ex- | Ex- | Ex- | Ex- |
|---|---|---|---|---|---|---|
| | ample | ample | ample | ample | ample | ample |
| Classification | 4 | 5 | 6 | 7 | 8 | 9 |
| Melting point | 75° C. | 80° C. | 85° C. | 85° C. | 97° C. | 100° C. |
| of mixture | | | | to | to | or |
| | | | | 90° C. | 100° C. | higher |

On the basis of the results of Table 8, it was found that the smaller the content ratio suitable for prescription, the better. In addition, the melting point of the composition is preferably in the range of 80° C. to 90° C. in terms of the preparation process and the high-temperature stability of the formulation. Accordingly, it was found that Example 7 in which the content of the gelling agent was lowest and the melting point was within a suitable range is most suitable. That is, it is preferable to use the gelling agent and the solute-to-solvent ratio used in Example 8.

### EXAMPLES 10 TO 14

Water-in-oil cosmetics of Examples 10 to 14 were prepared according to the compositions shown in Table 9 below.

TABLE 9

| Classification | Name of component | Example 10 | Example 11 | Example 12 | Example 13 | Example 14 |
|---|---|---|---|---|---|---|
| Gelling agent | Dibutyllauroyl | 0.400 | 0.800 | 1.000 | 1.500 | 2.000 |
| Mixture | glutamide | | | | | |
| | Octyldodecanol | 3.600 | 4.800 | 6.000 | 9.000 | 12.00 |
| Structuring | Microcrystalline wax | 2.000 | 2.000 | 2.000 | 2.000 | 2.000 |
| agent | Synthetic wax | 3.000 | 3.000 | 3.000 | 3.000 | 3.000 |
| | Disteadymonium | 0.540 | 0.540 | 0.540 | 0.540 | 0.540 |
| | hectorite | | | | | |
| Emulsifier | Cetyl PEG/PPG- | 1.500 | 1.500 | 1.500 | 1.500 | 1.500 |
| | 10/1 dimethicone | | | | | |
| | Polyglyceryl-4 | 1.000 | 1.000 | 1.000 | 1.000 | 1.000 |
| | Isostearate | | | | | |
| | Sorbitan Isostearate | 0.500 | 0.500 | 0.500 | 0.500 | 0.500 |
| Oil phase | Phenyltrimethicone | 10.00 | 10.00 | 10.00 | 10.00 | 10.00 |
| | Cyclopentasiloxane | 3.000 | 3.000 | 3.000 | 3.000 | 3.000 |
| | Triethylhexanoin | 5.000 | 5.000 | 5.000 | 5.000 | 5.000 |
| Pigment | Titanium dioxide | 10.000 | 10.000 | 10.000 | 10.000 | 10.000 |
| | Red iron oxide | 0.202 | 0.202 | 0.202 | 0.202 | 0.202 |
| | Yellow iron oxide | 0.842 | 0.842 | 0.842 | 0.842 | 0.842 |
| | Black iron oxide | 0.054 | 0.054 | 0.054 | 0.054 | 0.054 |
| Water-soluble | Disodium EDTA | 0.050 | 0.050 | 0.050 | 0.050 | 0.050 |
| component | Magnesium sulfate | 1.000 | 1.000 | 1.000 | 1.000 | 1.000 |
| Component | Purified water | Balance | Balance | Balance | Balance | Balance |
| | Glycerin | 8.000 | 8.000 | 8.000 | 8.000 | 8.000 |
| | Preservative | Appropriate | Appropriate | Appropriate | Appropriate | Appropriate |
| | | amount | amount | amount | amount | amount |

The state of each composition immediately after preparation thereof according to the content of the mixture (i.e., gelling agent) in each of Examples 10 to 14, and time-dependent changes of each composition according to temperature were observed. The results are shown in Table 10 below.

TABLE 10

| Classifi- | Storage temperature | | | | | Discharge test | |
|---|---|---|---|---|---|---|---|
| cation | 25° C. | 4° C. | 44° C. | 50° C. | Cycle | 44° C. | 50° C. |
| Exam- | ⊚ | ◯ | Δ | Δ | Δ | Abnor- | Abnor- |
| ple 10 | | | | | | mal | mal |
| Exam- | ⊚ | ⊚ | ◯ | ◯ | ◯ | Normal | Normal |
| ple 11 | | | | | | | |
| Exam- | ⊚ | ⊚ | ⊚ | ◯ | ◯ | Normal | Normal |
| ple 12 | | | | | | | |
| Exam- | ⊚ | ⊚ | ⊚ | ⊚ | ◯ | Normal | Normal |
| ple 13 | | | | | | | |
| Exam- | ⊚ | ◯ | Δ | X | Δ | Abnor- | Abnor- |
| ple 14 | | | | | | mal | mal |

⊚: very good,
◯: good,
Δ: slight separation,
X: separation

The optimal content of the gelling agent for a water-in-oil cosmetic composition can be determined on the basis of the results of Table 10, and it was found that Example 12 and Example 13 showed the optimal content on the basis of the storage temperatures and the results of the discharge tests. When the content of the gelling agent is lower than that of Example 11, the hardness is insufficient so that the cosmetic may be collapsed by a cutting blade at high temperature. On the other hand, as in Example 14, when the content of the gelling agent is higher than that of Example 13, since the melting point of the gelling agent increases, the emulsification becomes unstable. Therefore, the appropriate content of the gelling agent may be within a range of 0.5 wt % to 1.7 wt %.

### EXAMPLE 15 AND COMPARISON EXAMPLES 14 AND 15

Water-in-oil cosmetics of Example 15 and Comparative Example 4 and 5 were prepared according to the compositions shown in Table 11 below.

TABLE 11

| | Name of | | Comparative | Comparative |
|---|---|---|---|---|
| Classification | component | Example 15 | Example 4 | Example 5 |
| Gelling agent | Dibutyllauroyl | 1.500 | 1.500 | 1.500 |
| Mixture | glutamide | | | |
| | Octyldodecanol | 9.000 | 9.000 | 9.000 |
| Structuring | Microcrystalline | 2.000 | 2.000 | 2.000 |
| agent | wax | | | |
| | Synthetic wax | 3.000 | 3.000 | 3.000 |
| | Disteadymonium | 0.540 | 0.540 | 0.540 |
| | hectorite | | | |
| PEG-based | Cetyl PEG/PPG- | 0.300 | 0.100 | 1.000 |
| Emulsifier | 10/1 dimethicone | | | |
| | PEG-10 | 0.300 | 0.300 | 0.300 |
| | dimethicone/vinyl | | | |
| | dimethicone | | | |
| | crosspolymer | | | |
| Oil phase | Phenyltrimethicone | 10.00 | 10.00 | 10.00 |
| | Caprylic/capric | 2 | 2 | 2 |
| | triglycerides | | | |
| | Cyclopentasiloxane | 3.000 | 3.000 | 3.000 |
| | Triethylhexanoin | 5.000 | 5.000 | 5.000 |
| Pigment | Titanium dioxide | 10.000 | 10.000 | 10.000 |
| | Red iron oxide | 0.202 | 0.202 | 0.202 |
| | Yellow iron oxide | 0.842 | 0.842 | 0.842 |
| | Black iron oxide | 0.054 | 0.054 | 0.054 |
| Water-soluble | Disodium EDTA | 0.050 | 0.050 | 0.050 |
| component | Magnesium sulfate | 1.000 | 1.000 | 1.000 |
| | Purified water | Balance | Balance | Balance |
| | Glycerin | 8.000 | 8.000 | 8.000 |
| | Preservative | Appropriate | Appropriate | Appropriate |
| | | amount | amount | amount |

The state of each PEG-based water drop cosmetic immediately after preparation thereof according to the emulsifiers of Example 15 and Comparative Examples 4 and 5, and time-dependent changes of each cosmetic according to temperature were observed. The results are shown in Table 12 below.

TABLE 12

| | Storage temperature | | | | | Discharge test | | Water bursting |
|---|---|---|---|---|---|---|---|---|
| Classification | 25° C. | 4° C. | 44° C. | 50° C. | Cycle | 44° C. | 50° C. | phenomenon |
| Example 15 | ⊚ | ⊚ | ⊚ | ◯ | ◯ | Normal | Normal | ⊚ |
| Comparative | ◯ | Δ | Δ | X | Δ | Normal | Abnormal | ◯ |
| Example 4 | | | | | | | | |
| Comparative | ⊚ | ⊚ | ⊚ | ◯ | ◯ | Normal | Normal | Δ |
| Example 5 | | | | | | | | |

⊚: very good,
◯: good,
Δ: slight separation,
X: separation

On the basis of the results of Table 12, the optimal content of the PEG-based emulsifier in the water drop formulation was confirmed. In Comparative Example 4, since the content of the emulsifier was low, the water bursting effect was good, but the emulsified state was unstable. In Comparative Example 5, since the content of the emulsifier increased, the size of the emulsified particles decreased, and the water bursting effect was reduced. Therefore, the optimal content of the PEG-based emulsifier is composed of 0.2 wt % to 0.8 wt % of cetyl PEG/PPG-10/1 dimethicone and 0.2 wt % to 0.4 wt % of PEG-10 dimethicone/vinyl dimethicone crosspolymer.

### EXAMPLES 16 AND 17 AND COMPARATIVE EXAMPLES 6 TO 9

Water-in-oil cosmetics of Examples 16 and 17 and Comparative Examples 6 to 9 were prepared according to the compositions shown in Table 13 below.

TABLE 13

| | | | Comparative | Comparative | | Comparative | Comparative |
|---|---|---|---|---|---|---|---|
| Classification | Name of component | Example 16 | Example 6 | Example 7 | Example 17 | Example 8 | Example 9 |
| Gelling agent | Dibutyllauroyl | 0.600 | 0.600 | 0.600 | 1.000 | 1.500 | 2.000 |
| Mixture | glutamide | | | | | | |
| | Octyldodecanol | 3.600 | 3.600 | 3.600 | 6.000 | 9.000 | 12.00 |
| Structuring | Microcrystalline wax | 2.000 | 2.000 | 2.000 | 2.000 | 2.000 | 2.000 |
| agent | Synthetic wax | 3.000 | 3.000 | 3.000 | 3.000 | 3.000 | 3.000 |
| | Disteadymonium | 0.540 | 0.540 | 0.540 | | | |
| | Hectorite | | | | | | |
| | Stearalkonium | | | | 0.252 | 0.252 | 0.252 |
| | Hectorite | | | | | | |
| Polyglyceryl | Polyglyceryl-3 | 0.300 | 0.100 | 1.000 | | | |
| emulsifier | polydimethylsiloxy- | | | | | | |
| | ethyldimethicone | | | | | | |
| | Polyglyceryl-3 | | | | 0.600 | 0.300 | 1.000 |
| | polyricinoleate | | | | | | |
| | Polyglyceryl-3 | | | | 0.120 | 0.120 | 0.120 |
| | Diisostearate | | | | | | |
| Oil phase | Phenyltrimethicone | 10.00 | 10.00 | 10.00 | 10.00 | 10.00 | 10.00 |
| | Caprylic/Capric | 2.000 | 2.000 | 2.000 | 2.000 | 2.000 | 2.000 |
| | Triglycerides | | | | | | |
| | Cyclopentasiloxane | 3.000 | 3.000 | 3.000 | 3.000 | 3.000 | 3.000 |
| | Triethylhexanoin | 5.000 | 5.000 | 5.000 | 5.000 | 5.000 | 5.000 |
| Pigment | Titanium dioxide | 10.00 | 10.00 | 10.00 | 10.00 | 10.00 | 10.00 |
| | Red iron oxide | 0.200 | 0.200 | 0.200 | 0.202 | 0.202 | 0.202 |
| | Yellow iron oxide | 0.840 | 0.840 | 0.840 | 0.842 | 0.842 | 0.842 |
| | Black iron oxide | 0.050 | 0.050 | 0.050 | 0.054 | 0.054 | 0.054 |
| Water-soluble | Disodium EDTA | 0.050 | 0.050 | 0.050 | 0.050 | 0.050 | 0.050 |
| component | Magnesium sulfate | 1.000 | 1.000 | 1.000 | 1.000 | 1.000 | 1.000 |
| | Purified water | Balance | Balance | Balance | Balance | Balance | Balance |
| | Glycerin | 8.000 | 8.000 | 8.000 | 8.000 | 8.000 | 8.000 |
| | Preservative | Appropriate | Appropriate | Appropriate | Appropriate | Appropriate | Appropriate |
| | | amount | amount | amount | amount | amount | amount |

The state of each PEG-based water drop cosmetic immediately after preparation thereof according to polyglyceryl emulsifiers of Examples 16 and 17 and Comparative Examples 6 to 9, and time-dependent changes of each cosmetic according to temperature were observed. The results are shown in Table 14 below.

TABLE 14

| | Storage temperature | | | | | Discharge test | | Water bursting |
|---|---|---|---|---|---|---|---|---|
| Classification | 25° C. | 4° C. | 44° C. | 50° C. | Cycle | 44° C. | 50° C. | phenomenon |
| Example 16 | ⊚ | ⊚ | ⊚ | ◯ | ◯ | Normal | Normal | ⊚ |
| Comparative | ◯ | Δ | Δ | X | Δ | Abnormal | Abnormal | ◯ |
| Example 6 | | | | | | | | |
| Comparative | ⊚ | ⊚ | ⊚ | ⊚ | ◯ | Normal | Normal | Δ |
| Example 7 | | | | | | | | |
| Example 17 | ⊚ | ⊚ | ◯ | ◯ | ◯ | Normal | Normal | ⊚ |
| Comparative | ◯ | Δ | Δ | X | Δ | Abnormal | Abnormal | ◯ |
| Example 8 | | | | | | | | |
| Comparative | ⊚ | ⊚ | ⊚ | ◯ | ◯ | Normal | Normal | Δ |
| Example 9 | | | | | | | | |

⊚: very good,
◯: good,
Δ: slight separation,
X: separation

On the basis of the results of Table 14, the optimal content of the polyglyceryl emulsifier in the water drop formulation was confirmed. In Comparative Examples 6 and 8, since the content of the emulsifier was low, the water bursting effect was good, but the emulsified state was unstable. In Comparative Examples 7 and 9, since the content of the emulsifier increased, the size of the emulsified particles decreased, and the water bursting effect was reduced. Therefore, the optimal content of the polyglyceryl emulsifier is composed of 0.2 wt % to 0.8 wt % of polyglyceryl-3 polydimethylsiloxyethyldimethicone and 0.1 wt % to 0.2 wt % of polyglyceryl-3 diisostearate, or 0.4 wt % to 0.8 wt % of Polyglyceryl-3 polyricinoleate and 0.1 wt % to 0.2 wt % of glyceryl-3 diisostearate.

### EXAMPLES 18 TO 22

Oil dispersion cosmetics of Examples 18 to 22 were prepared according to the compositions shown in Table 15 below.

TABLE 15

| Classification | Name of component | Example 18 | Example 19 | Example 20 | Example 21 | Example 22 |
|---|---|---|---|---|---|---|
| Gelling agent | Dibutyllauroyl | 0.600 | 0.800 | 1.000 | 1.500 | 2.000 |
| Mixture | glutamide | | | | | |
| | Octyldodecanol | 3.600 | 4.800 | 6.000 | 9.000 | 12.00 |
| Structuring | Microcrystalline wax | 2.000 | 2.000 | 2.000 | 2.000 | 2.000 |
| agent | Synthetic wax | 3.000 | 3.000 | 3.000 | 3.000 | 3.000 |
| | Disteadymonium | 0.540 | 0.540 | 0.540 | 0.540 | 0.540 |
| | hectorite | | | | | |
| Emulsifier | Cetyl PEG/PPG- | 0.300 | 0.300 | 0.300 | 0.300 | 0.300 |
| | 10/1 dimethicone | | | | | |
| Oil phase | Phenyltrimethicone | 10.00 | 10.00 | 10.00 | 10.00 | 10.00 |
| | Cyclopentasiloxane | 3.000 | 3.000 | 3.000 | 3.000 | 3.000 |
| | Triethylhexanoin | 5.000 | 5.000 | 5.000 | 5.000 | 5.000 |
| Pigment | Titanium dioxide | 10.000 | 10.000 | 10.000 | 10.000 | 10.000 |
| | Red iron oxide | 0.202 | 0.202 | 0.202 | 0.202 | 0.202 |
| | Yellow iron oxide | 0.842 | 0.842 | 0.842 | 0.842 | 0.842 |
| | Black iron oxide | 0.054 | 0.054 | 0.054 | 0.054 | 0.054 |
| Powder | Silica | 10.00 | 10.00 | 10.00 | 10.00 | 10.00 |
| | Mica | Balance | Balance | Balance | Balance | Balance |

The state of each composition immediately after preparation thereof according to the content of the mixture (i.e., gelling agent) in each of Examples 18 to 22, and time-dependent changes of each composition according to temperature were observed. The results are shown in Table 16 below.

TABLE 16

| Classifi- | Storage temperature | | | | | Discharge test | |
|---|---|---|---|---|---|---|---|
| cation | 25° C. | 4° C. | 44° C. | 50° C. | Cycle | 44° C. | 50° C. |
| Exam- | ⊚ | ◯ | Δ | Δ | Δ | Normal | Abnor- |
| ple 18 | | | | | | | mal |
| Exam- | ⊚ | ⊚ | ⊚ | ⊚ | ⊚ | Normal | Normal |
| ple 19 | | | | | | | |
| Exam- | ⊚ | ⊚ | ⊚ | ⊚ | ⊚ | Normal | Normal |
| ple 20 | | | | | | | |
| Exam- | ⊚ | ⊚ | ⊚ | ◯ | Δ | Normal | Abnor- |
| ple 21 | | | | | | | mal |
| Exam- | ⊚ | ◯ | Δ | X | Δ | Abnor- | Abnor- |
| ple 22 | | | | | | mal | mal |

⊚: very good,
◯: good,
Δ: slight separation,
X: separation

The optimal content of the gelling agent for an oil dispersion cosmetic composition can be determined on the basis of the results of Table 16, and it was found that Example 19 and Example 20 showed the optimal content, on the basis of the storage temperatures and the results of the discharge tests.

According to the embodiments of the present disclosure, the solid cosmetic composition with improved high-temperature stability and good feeling of use and the preparation method thereof can provide a cosmetic that can maintain the structure thereof even at a high temperature of about 50° C. and thus has high temperature stability due to an increased melting point compared to existing cosmetic compositions. In addition, the cosmetic prepared from the cosmetic composition according to the present disclosure can withstand not only thermal stress at high temperatures but also physical stress caused by external forces.

In addition, even when the cosmetic is contained in a grinding container, the cosmetic can be readily shaved and discharged by a rotating cutting blade. The cosmetic does not give heavy feeling while having an increased hardness and can be lightly and thinly applied to the skin.

In addition, the cosmetic can provide a thin and soft feeling of use, thereby giving natural skin expression which is a recent cosmetic trend. In addition, since a small amount of a gelling agent is used to improve the hardness of a cosmetic instead of existing structuring agents composed of only wax-based materials, it is possible to increase the degree of freedom in designing a cosmetic composition.

Although the present disclosure has been described above with reference to the examples, it will be appreciated that those skilled in the art variously modify and change the examples of the present disclosure without departing from the spirit and scope of the present invention as set forth in the appended claims. Therefore, when modifications to the examples includes the elements of the claims of the present disclosure, it should be considered that all of the modifications fall within the technical scope of the present disclosure.

What is claimed is:

1. A composition for a solid cosmetic with improved high-temperature stability and good feeling of use, the composition containing an oily gelling agent.

2. The composition according to claim 1, wherein the oily gelling agent comprises at least one selected from the group consisting of dibutylethylhexanoylglutamide, dibutyllauroylglutamide, dextrin palmitate, and mixtures thereof.

3. The composition according to claim 2, wherein when the oily gelling agent is dibutylethylhexanoylglutamide, the dibutylethylhexanoylglutamide is contained in an amount of 0.5 wt % to 1.7 wt % relative to the total weight of the composition.

4. The composition according to claim 2, wherein when the oily gelling agent is dibutyllauroylglutamide, the dibutyllauroylglutamide is contained in an amount of 0.5 wt % to 1.7 wt % relative to the total weight of the composition.

5. The composition according to claim 2, wherein when the oily gelling agent is a mixture of dibutylethylhexanoylglutamide and dibutyllauroyl glutamide, the dibutylethylhexanoylglutamide and the dibutyllauroyl glutamide are contained in an amount of 0.25 wt % to 0.85 wt % and an amount of 0.25 wt % to 0.85 wt %, respectively, with respect to the total weight of the composition.

6. The composition according to claim 2, wherein the oily gelling agent is prepared by using octyldodecanol that is an aliphatic alcohol as a solvent.

7. The composition according to claim 6, wherein the content of a solute of the oily gelling agent is 5 to 9 times the content of a solvent, and the solute is dissolved at a temperature of 110° C. to 120° C.

8. The composition according to claim 1, further containing an oil-soluble component,
wherein the oil-soluble component comprises at least one selected from the group consisting of fat, wax, hydrocarbon, natural oil, ester, hydrocarbon, silicone, organic UV absorber, and mixtures thereof.

9. The composition according to claim 8, wherein the oil-soluble component is contained in an amount of 20% to 60% relative to the total weight of the composition.

10. The composition according to claim 1, wherein the composition for a solid cosmetic is a water-in-oil type cosmetic composition, an emulsifier contained in the composition comprises
at least one selected from the group consisting of cetyl PEG/PPG-10/1 dimethiocone, PEG-10 dimethicone/vinyl dimethicone crosspolymer, polyglyceryl-3 polydimethylsiloxyethyl dimethicone, polyglyceryl-3 polyricinoleate, polyglyceryl-3 diisostearate, and mixtures thereof.

11. The composition according to claim 10, wherein the cetyl PEG/PPG-10/1 dimethiocone and the PEG-10 dimethicone/vinyldimethicone crosspolymer are contained in an amount of 0.2 wt % to 0.8 wt % and an amount of 0.2 wt % to 0.4 wt %, respectively.

12. The method according to claim 10, wherein the polyglyceryl-3 polydimethylsiloxyethyl dimethicone is contained in an amount of 0.2 wt % to 0.8 wt %.

13. The method according to claim 10, wherein the polyglyceryl-3 polyricinoleate and the polyglyceryl-3 diisostearate are contained in an amount of 0.4 wt % to 0.8 wt % and an amount of 0.1 wt % to 0.2 wt %, respectively.

14. A method of preparing a composition for a solid cosmetic with improved high-temperature stability and good feeling of use, the method comprising:
mixing an oily gelling agent, aliphatic alcohol, ester oil, and pigment base that are oily components for a first predetermined time at a stirring rate of 900 rpm to 1100 rpm using a mixer at a high temperature;
adding a water-soluble component to the mixture of the oily components to prepare an emulsion while mixing the mixture of the oily components at a high temperature of 75° C. to 80° C. using a homomixer at a stirring rate of 1400 rpm to 1600 rpm for a second predetermined time; and
adding a powder component including a pigment and an additive to the emulsion and further mixing at a stirring rate of 1400 rpm to 1600 rpm for a third predetermined time.

15. The method according to claim 14, wherein an emulsifier used to prepare the emulsion comprises
at least one selected from the group consisting of cetyl PEG/PPG-10/1 dimethiocone, PEG-10 dimethicone/vinyl dimethicone crosspolymer, polyglyceryl-3 polydimethylsiloxyethyl dimethicone, polyglyceryl-3 polyricinoleate, polyglyceryl-3 diisostearate, and mixtures thereof.

16. A method of preparing a composition for a solid cosmetic with improved high-temperature stability and good feeling of use, the method comprising:
mixing an oily gelling agent, aliphatic alcohol, ester oil, and pigment base that are oily components for a first predetermined time at a stirring rate of 900 rpm to 1100 rpm using a mixer at a high temperature:
further mixing the resulting mixture obtained through the previous mixing at a high temperature of 75° C. to 80° C. using a homomixer at a stirring rate of 1400 rpm to 1600 rpm for a second predetermined time; and
adding a pigment, a powder component, and an additive to the mixture obtained through the previous mixing and further mixing at a stirring rate of 1400 rpm to 1600 rpm for a third predetermined time.
